(19) United States
(12) Patent Application Publication
(10) Pub. No.: US 2005/0255144 A1
(43) Pub. Date: Nov. 17, 2005

(54) Methods and articles for the delivery of medicaments to the eye for the treatment of posterior segment diseases

(75) Inventor: Clyde Schultz, Ponte Vedra, FL (US)

Correspondence Address: FINCH IP LLC, P.O. BOX 1358, CONCORD, NH, 03302, US

(73) Assignee: DirectContact LLC

(21) Appl. No.: 11/102,454

(22) Filed: Apr. 9, 2005

Related U.S. Application Data
(63) Continuation-in-part of application No. 10/971,997, filed on Oct. 22, 2004.
Application No. 10/971,997 is a continuation-in-part of application No. 10/821,718, filed on Apr. 9, 2004.
(60) Provisional application No. 60/461,354, filed on Apr. 9, 2003.

(51) Int. Cl. A61F 2/00

(52) U.S. Cl. 424/428

(57) ABSTRACT

This invention provides articles and methods for drug delivery including a hydrogel containing one or more drugs for the treatment of a posterior segment disease and/or dry eye conditions. Exemplary drugs are anti-angiogenesis compounds for the treatment of macular degeneration. Allowing passive transference of this drug from a dilute solution into the hydrogel produces the delivery system. The hydrogel, when placed in contact with the eye, delivers the drug. The delivery of the drug is sustained over an extended period of time, which is of particular utility in the eye, which is periodically flushed with tears. This sustained delivery accelerates the treatment process while avoiding potential damaging effects of localized delivery of high concentrations of compounds, e.g., from eye drops.

### CROSS-REFERENCE TO RELATED APPLICATIONS

This application is a continuation-in-part of U.S. application Ser. No. 10/971,997, filed Oct. 22, 2004 which is a continuation-in-part of U.S. application Ser. No. 10/821,718, filed Apr. 9, 2004, which claims benefit of U.S. Provisional Application No. 60/461,354, filed Apr. 9, 2003, each of which is hereby incorporated by reference.

### BACKGROUND OF THE INVENTION

1. Field of the Invention

This application relates to methods and articles for the treatment of eye conditions and, in particular, to the delivery of medicaments for the treatment of posterior segment diseases.

2. Related Art

Posterior Segment Diseases of the eye, also known as back of eye (BOE) diseases, include diseases such as age-related macular degeneration (AMD) and vascular retinopathy. AMD is often characterized as one of two types, either “wet” or “dry.” While dry AMD is the most prevalent, the wet form is typically more serious and can lead to blindness. The dry form can advance to the wet form in some cases. Drugs exist for the treatment of posterior segment diseases and new ones are being developed and tested. However, for a variety of reasons, delivery of drugs to the posterior segment can be difficult, dangerous or painful.

Systemic and topical (e.g., via eye drops) administration of drugs for treatment of diseases of the posterior segment of the eye is often undesirable. These methods typically require high total doses of the drug because these routes are inefficient at delivering the drug to the posterior segment. Such high doses increase the cost and may also cause side effects such as local inflammation or adverse systemic reactions. In addition, for many topical treatments, unknown amounts of the drug are quickly washed out of the eye, limiting the effective time of treatment. Thus, the amount of drug reaching the posterior segment may be difficult to gauge and may vary greatly between subjects.

“Dry eye” is characterized by a lack of moisture and/or lubrication in the eye due to, for example, inadequate tear production or inadequate moisture retention. Symptoms include scratchiness and burning in the eye and in some cases the cornea can be damaged if dry eye is not treated. Dry eye is a growing concern and is particularly pronounced in the portion of the population over age 65. Dry eye may be temporary or chronic and may accompany other diseases of the eye, such as posterior segment diseases. Dry eye may also be the result of hormonal changes and/or autoimmune disease. Dry eye is not in itself a disease but is a sign of a disease or other disorder. Some conditions often associated with dry eye include, for example, rheumatoid arthritis, Sjogren's syndrome, keratoconjunctivitis sicca, xerophthalmia, lupus erythematosis, Grave's disease, diabetes, or scleroderma.

Dry eye treatment has typically included artificial tears, or other ways of adding moisture to the eye. Therapeutic treatments, those that increase tear production in the eye, also exist, but efficient delivery of these drugs suffers from some of the same problems as do the posterior segment drugs. For instance, the drugs cannot be administered at constant doses for extended times.

In U.S. Pat. No. 5,723,131 a method is described in which desferrioxamine is leached from a contact lens into the ocular fluid of the eye to prevent the growth of bacteria in the ocular fluid.

Sustained-release delivery devices that continuously administer a drug to the eye for a prolonged period of time are desired for the treatment of posterior segment diseases and for dry eye disorders.

### SUMMARY OF THE INVENTION

The present invention relates to articles, such as hydrogel drug delivery systems, and methods of producing and using such articles for the treatment of disease in the posterior segment of the eye, e.g., the vitreous, retina (including the macula), choroids, sclera, and optic nerve. Also included are articles and methods for the treatment of dry eye conditions. The articles may include a substrate, such as a hydrogel, into which one or more drugs are transferred from a dilute solution, e.g., an aqueous solution. When placed in contact with eye tissue, the drug or drugs passively transfer out of the hydrogel to provide treatment of posterior segment diseases or dry eye disorders.

In one aspect, an article for treatment of posterior segment eye disease is provided, the article comprising a substrate and a drug wherein the article is capable of placement on the eye and the drug is capable of being passively released from the article.

In another aspect a method of making an article for delivery of a drug for the treatment of posterior segment eye disease is provided, the method comprising providing a substrate that is conforming or conformable to the surface of a human eye, and absorbing a posterior segment drug into the substrate.

In another aspect a method of treating a subject having or at risk of having posterior segment eye disease is provided, the method comprising providing an article that comprises a substrate and a drug, placing the article in contact with the subject's eye, and allowing the drug to be transported from the article to the eye.

In another aspect, a method of treating a subject having or at a risk of having posterior segment eye disease is provided, the method comprising administering to the subject a pharmaceutically effective quantity of a VEGF ligand consisting essentially of a nucleic acid.

In another aspect, an article for therapeutic treatment of a dry eye condition is provided, the article comprising a substrate and a drug wherein the article is capable of placement on the eye and the drug is capable of being passively released from the article.

In another aspect, a method of treating a subject having or at risk of having a dry eye condition is provided, the method comprising providing an article that comprises a substrate and a dry eye drug, placing the article in contact with the subject's eye, and releasing the drug from the article.

Other features and advantages of the invention will be apparent from the following figures, description and the claims.

### BRIEF DESCRIPTION OF DRAWINGS

In the drawing,

FIGS. 1A and 1B are photomicrographs of histological slides of retinal tissue from untreated (1A) and treated (1B) samples.

### DETAILED DESCRIPTION OF THE INVENTION

This invention provides a drug delivery system that may include a hydrogel containing one or more drugs for the treatment of a posterior segment disease. Allowing passive transference of this drug from a dilute solution into the hydrogel produces the delivery system. The hydrogel, when placed in contact with the eye, delivers the drug. The delivery of the drug is sustained over an extended period of time, which is of particular utility in the eye, which is periodically flushed with tears. This sustained delivery may accelerate the treatment process while avoiding potential damaging effects of localized delivery of high concentrations of drugs compared, e.g., to eye drops. Drugs can be administered non-systemically and with a non-penetrating medical device.

Accordingly, in one aspect, the invention features an article that contains a drug for the treatment of a posterior segment disease, wherein the drug is capable of being passively released in a therapeutically effective amount to treat the posterior segment disease. The article may be placed in contact with the eye for an extended period of time, e.g., greater than 1 minute, greater than 1 hour, or greater than 1 day. The article may be any material that can be placed on, or in contact with, the eye for an extended period of time. The article may be placed on the cornea and/or limbus and/or sclera. Suitable materials for the article include polymers, hydrogels, polymeric hydrogels and contact lens materials. Preferably, the materials are hydrophilic.

The article may be of any size that can be placed on the eye and may, for instance, have a diameter greater than or less than 1 mm, 2 mm, 3 mm, 5 mm or 10 mm. Larger sized articles will generally have a greater loading capacity which is typically a factor of the volume of the article as well as its composition. It may be transparent and be of any shape although typically the article is substantially round. The article may be of any thickness such as, for example, greater than 0.1 mm, greater than 0.5 mm or greater than 1 mm.

In some cases, the article may dissolve or break down in situ. In other embodiments, the article is not degradable and may remain intact for greater than 1 day, greater than one month or greater than one year. If an article is to be re-used by re-loading it with a drug or drugs it is preferred that the article is not degradable. More than one article may be placed in an eye but typically a single article is adequate for delivering a desired supply of medicament.

Exemplary hydrogel materials include a tetrapolymer of hydroxymethylmethacrylate, ethylene glycol, dimethylmethacrylate, and methacrylic acid. Other examples of hydrogels include etafilcon A, vifilcon A, lidofilcon A, vasurfilcon A, and polymacon B. In addition, variations of these polymers formed by the use of different packing solutions (e.g., phosphate-buffered saline and boric acid) in the manufacturing process are also included. The hydrogel may be ionic or non-ionic. In various embodiments, the drug is capable of being passively released into the ocular environment under ambient or existing conditions. In other embodiments, the hydrogel may be shaped as a contact lens, e.g., one capable of correcting vision. Such a contact lens may be capable of correcting vision in the range of +8.0 to −8.0 diopters or may be plano. The contact lens may also have a base curve between 8.0 and 9.0.

The invention further features a method for making a hydrogel drug delivery system by placing the hydrogel, e.g., a contact lens, in a solution containing one or more drugs as described herein, which can be passively transferred to the hydrogel. This method may further include the steps of washing the hydrogel in an isotonic saline solution and partially desiccating the hydrogel prior to placement in the solution. The solution may have, e.g., a pH between 6.9 and 7.4, and a drug concentration of between 0.00001 and 10%. In one embodiment, the hydrogel is placed in the solution of drug for at least 30 minutes.

In another aspect, the invention features a method for treating a posterior segment disease. The method includes placing a hydrogel, as described herein, in contact with an eye, wherein the drug or drugs are passively released from the hydrogel to treat the disease. In various embodiments, the posterior segment disease is in the vitreous, retina (e.g., the macula), choroids, sclera, or optic nerve. The hydrogel may passively release, for example, at least 0.0001, 0.0005, 0.001, 0.005, 0.01, 0.05, 0.1, 0.5, 1, 10, 15, 20, 50, 75, 100, 250, 500, or 1000 μg of a drug, and the hydrogel may be placed in contact with the eye for at least 0.5, 1, 1.5, 2, 2.5, 3, 3.5, 4, 4.5, 5, 7.5, 10, 15, or 24 hours. The method for treating a posterior segment disease, e.g., macular degeneration, may further include the step of diagnosing the posterior segment disease prior to placing the hydrogel in contact with the eye.

Exemplary drugs and posterior segment diseases are described herein. Preferred drugs include anti-angiogenesis compounds, as described herein, for the treatment of macular degeneration.

As used herein, by “ambient condition(s)” is meant room temperature and pressure or temperature and pressure at the surface of the eye.

In “contact” with the eye means that the article is placed on the surface of the eye. There may be a layer of ocular fluid, e.g., saline, tears or mucin, between the article and the eye tissue (cornea).

By “existing conditions” is meant in situ in the eye.

By “treating” is meant medically managing a patient with the intent that a prevention, cure, stabilization, or amelioration of the symptoms will result. This term includes active treatment, that is, treatment directed specifically toward improvement of the disease; palliative treatment, that is, treatment designed for the relief of symptoms rather than the curing of the disease; preventive treatment, that is, treatment directed to prevention of the disease; and supportive treatment, that is, treatment employed to supplement another specific therapy directed toward the improvement of the disease. The term “treating” also includes symptomatic treatment, that is, treatment directed toward constitutional symptoms of the disease.

By “ocular environment” is meant the tissues of and surrounding the eye, including, for example, the sclera, cornea, and other tissues of the ocular cavity and the posterior segment.

The “posterior segment” of the eye includes, without limitation, the vitreous, retina (including the macula), choroids, sclera, and optic nerve.

Exemplary posterior segment diseases include, without limitation, retinal detachment, diabetic retinopathy, macular degeneration (e.g., age-related), proliferative vitreoretinopathy, endophthalmitis, retinopathy of prematurity, posterior segment trauma, intraocular lens-related posterior segment complications, retinal vascular diseases, macular edema, intraocular tumors, hereditary retinal degenerations, AIDS-related retinitis, posterior segment uveitis, and systemic diseases with retinal manifestations. For the purposes of this invention, glaucoma is not a posterior segment disease.

Exemplary dry eye conditions include, without limitation, rheumatoid arthritis, Sjogren's syndrome, keratoconjunctivitis sicca, xerophthalmia, lupus erythematosis, Grave's disease, diabetes, or scleroderma.

All percentages described in the present invention are by weight unless otherwise specified.

Posterior Segment Diseases

Posterior segment diseases to be treated by the present invention include, for example, retinal detachment, neovascularization, diabetic retinopathy, macular degeneration (e.g., age-related), proliferative vitreoretinopathy, endophthalmitis, retinopathy of prematurity, posterior segment trauma, intraocular lens-related posterior segment complications, retinal vascular diseases, macular edema (e.g., diabetic), intraocular tumors, retinal degeneration (e.g., hereditary), vascular retinopathy, inflammatory diseases of the retina, AIDS-related retinitis, uveitis, and systemic diseases with retinal manifestations. Neovascularizations include retinal, choroidal, and vitreal. The retinal neovascularization to be treated can be caused by diabetic retinopathy, vein occlusion, sickle cell retinopathy, retinopathy of prematurity, retinal detachment, ocular ischemia, or trauma. The intravitreal neovascularization to be treated can be caused by diabetic retinopathy, vein occlusion, sickle cell retinopathy, retinopathy of prematurity, retinal detachment, ocular ischemia, or trauma. The choroidal neovascularization to be treated can be caused by retinal or subretinal disorders of age-related macular degeneration, diabetic macular edema, presumed ocular histoplasmosis syndrome, myopic degeneration, angioid streaks, or ocular trauma. Other posterior segment diseases are known in the art.

Drug Delivery System

In one aspect, the invention relates to an article that contains a drug for the treatment of a posterior segment disease, wherein the drug is capable of being passively released in a therapeutically effective amount to treat the posterior segment disease. The article may be placed in contact with the eye for an extended period of time, e.g., greater than 1 minute, greater than 1 hour, or greater than 1 day. The article may be any material that can be placed on, or in contact with, the eye for an extended period of time. Suitable materials include polymers, hydrogels, polymeric hydrogels and contact lenses. Preferably, the materials are hydrophilic.

In one embodiment, the article can be a hydrogel such as a contact lens. Treatment of hydrogels, as described herein, is also applicable to other articles that can be used, unless otherwise noted. In one set of embodiments, conventional soft contact lenses can be used and can be either ionic or non-ionic hydrogels containing between 10% and 90%, e.g., 24% or 37.5% to 65% or 75%, water by weight and can have any base curve appropriate for the subject, e.g., from 8.0 to 9.0. The contact lenses may also have the ability to correct vision, for example, over a range of diopters of +8.0 to −8.0. Exemplary hydrogel contact lens materials include etafilcon A, vifilcon A, lidofilcon A, polymacon B, vasurfilcon A, and a tetrapolymer of hydroxymethylmethacrylate, ethylene glycol, dimethylmethacrylate, and methacrylic acid. These materials may also be employed in other physical forms. Other suitable hydrogel materials are known to those skilled in the art. The hydrogels may be insoluble, may absorbable (e.g., dissolve or degrade) over time in vivo, e.g., over one day, one week, one month, six months, or one year or more, or may be partially soluble and partially insoluble. The drug is passively delivered, for example, by diffusion out of the hydrogel, by desorption from the hydrogel, or by release as the hydrogel dissolves. Exemplary soluble materials include a copolymer of trimethylene carbonate and polyglycolicacid (e.g., Maxon), polyglactin 910 (e.g., Vicryl), glyconate (e.g., Monosyn), poly-p-dioxanone (e.g., Monoplus), polyglycolic acid (e.g., Safil), polyglycolic acid felt (e.g., Neoveil), poly-4-hydroxybutyrate, combinations of poly(L-lactide) and poly(L-lactide-co-glycolide), glycol methacrylate, poly-DL-lactide, and Primacryl (Johnson & Johnson, e.g., Craniosorb AFS). Exemplary materials that are partially soluble or degradable include a composite mesh of oxidized regenerated cellulose, polypropylene, and polydioxanone (e.g., Proceed Mesh from Ethicon) and a mesh of polypropylene (prolene) and poligelcaprone (Monocryl) (e.g., Ultrapro from Ethicon).

The drug delivery system may be produced from a partially desiccated hydrogel (or equivalently a partially hydrated hydrogel). The desiccation step removes, for example, approximately 5%, 10%, 15%, 20%, 25%, 30%, 40%, 50%, 60%, or 75% of the water in the hydrogel. Desiccation can occur, for example, by exposure of the hydrogel to ambient or humidity controlled air, by heating the hydrogel for a specific period of time, or by blowing dried gas, such as N₂, over the hydrogel. In one embodiment, the hydrogel is saturated with physiological (isotonic) saline prior to desiccation. The partially desiccated hydrogel can then be soaked, e.g., for at least 30 minutes, in a dilute solution of drug, e.g., at a pH between 6.9 to 7.4. In certain embodiments, the drug is transferred to a contact lens from a non-aqueous solvent, e.g., dimethyl sulfoxide, which may be at least partially removed and exchanged with an aqueous solution prior to use in a patient. The hydrogels may also be soaked in drug solution for at least 1 hour, 6 hours, 12 hours, or 24 hours. The concentration of drug into which the hydrogel is placed is typically 0.000001, 0.000005, 0.00001, 0.00005, 0.0001, 0.0005, 0.001, 0.005, 0.01, 0.05, 0.1, 0.5, 1, 5, 10, 15, 20, 50, 75, 100, 250, 500, or 1000 μg/mL. Higher concentrations may also be used, for example, to reduce the soaking time. The drug is typically passively transferred into the hydrogel. This transfer may occur at least in part by rehydrating the hydrogel in the presence of the drug. Diffusion of the drug into the water or polymer in the hydrogel may also occur. In alternative embodiments, a fully hydrated or fully desiccated hydrogel is placed in the soaking solution to produce the medicated hydrogel. In some embodiments, the transferring of a drug or drugs into a hydrogel or other article can be enhanced by, for example, heating, agitating or applying ultrasound.

Desirably, the concentration of drug transferred to the hydrogel is typically substantially lower than the solution in which the hydrogel is soaked. For example, the concentration of drug in the hydrogel is at least 2×, 5×, or 10× less than that of the soaking solution. Some drugs, however, may have a higher affinity for a hydrogel than the soaking solution, and such a hydrogel may have a higher concentration of drug than the solution in which it was soaked, e.g., at least 2×, 5×, or 10× more. The water content and type of hydrogel, time and conditions, e.g., temperature of soaking, composition of the soaking solution (e.g., ionic strength and pH), and type of drug employed also may influence the concentration of drug in the drug delivery system. Since the water content of the hydrogel may also help to determine the total amount of drug present in a hydrogel, it represents a variable by which to control the amount of drug delivered to a tissue. The production of a hydrogel containing a specified amount of drug can be accomplished by routine experimentation by one skilled in the art. Some factors that may influence a chosen amount of loading are a desired dose rate, the release rate of the drug in situ, and an anticipated contact time between the hydrogel and the subject's eye.

It is notable that in many embodiments a drug compound can be administered without administering systemically and/or without administering via intravitreal injection. In some instances, the drug may be absorbed by the lymphatic system or the circulatory system of the eye. However, the drug may remain isolated from other portions of the subject's body as the sinal cavity is protected by the blood brain barrier (BBB). Compounds may pass from the hydrogel into ocular fluid bathing the eye and from there may enter the eye, for example, by transport across the cornea or sclera or via the sinal cavity. From the vitreous or sinal cavity, the drug can reach its target, e.g., the retina, in the posterior segment. These paths of delivery can help to reduce the amount of dilution that a drug is subjected to as well as the amount of time that it takes for the drug to reach its intended target, e.g., the posterior segment or a portion thereof. As a result, drugs that may have been broken down or degraded if administered systemically can be administered in lower doses and in forms that need not be derivitized in order to achieve physiological stability.

Drugs for the Treatment of Posterior Segment Diseases.

Any drug for the treatment of a posterior segment disease may be included in a drug delivery system described herein. Classes of drugs include anti-infectives (e.g., antibiotics, antibacterial agents, antiviral agents, and antifungal agents); analgesics; anesthetics; antiallergenic agents; mast cell stabilizers; steroidal and non-steroidal anti-inflammatory agents; decongestants; antioxidants; nutritional supplements; angiogenesis inhibitors; antimetabolites; fibrinolytics; neuroprotective drugs; angiostatic steroids; mydriatics; cyclopegic mydriatics; miotics; vasoconstrictors; vasodilators; anticlotting agents; anticancer agents; antisense agents, immunomodulatory agents; carbonic anhydrase inhibitors; integrin antagonists; cyclooxgenase inhibitors; differentiation modulator agents; sympathomimetic agents; VEGF antagonists; immunosuppresant agents; and combinations and prodrugs thereof. Other suitable drugs are known in the art.

Exemplary drugs include 17-ethynylestradiol, 2-ethoxy-6-oxime-estradiol, 2-hydroxyestrone, 2-propenyl-estradiol, 2-propynl-estradiol, 4,9(11)-pregnadien-17α,21-diol-3,20-dione, 4,9(11)-pregnadien-17α,21-diol-3,20-dione-21-acetate, 4-methoxyestradiol, 5-fluorouracil, 6-mannosephosphate, acetazolamide, acetohexamide, acetylcholinesterase inhibitors, acyclovir, adrenal corticalsteroids, adriamycin, aldesleukin, aldose reductase inhbitors, alkylating agents including cyclophosphamide, alpha-tocopherol, amifostine, amphotericin B, anastrozole, anecortave acetate, angiostatic steroids, angiostatin, antazoline, anthracycline antibiotics, antibody to cytokines, anticlotting activase, anti-cytomegalovirus agents, antifibrinogen, antineogenesis proteins, arsenic trioxide, asparaginase, atenolol, atropine sulfate, azacytidine, azathioprine, AZT, bacitracin, bacitracin, betamethasone, betaxolol, bexarotene, bleomycin, busulfan, calcium channel antagonists (e.g., imodipine and diltiazem), capecitabine, carbachol, carmustine, cephalosporin antibiotics, chlorambucil, chloramphenicol, chlorpheniramine, chlorpropamide, chlortetracycline, colchicine, cyclooxgenase II inhibitors, cyclopentolate, cyclophosphamide, cyclosporine, cyclosporine A, cytarabine, cytochalasin B, cytokines, dacarbazine, dactinomycin, daunorubicin, demecarium bromide, dexamethasone, diamox, dichlorphenamide, didanosine, dihydroxylipoic acid, diisopropylfluorophosphate, docetaxel, echinocandin-like lipopeptide antibiotics, echothiophateiodide, eliprodil, endostatin, epinephrine, epirubicin hydrochloride, erythromycin, erythropoietin, eserine salicylate, estradiol, estramustine, etanercept, ethisterone, etoposide, etoposide phosphate, etretinate, eucatropine, exemestrane, famvir, fibrinolysin, filgrastim, floxuridine, fluconazole, fludarabine, fluocinolone, fluoromethalone, fluoroquinolone, fluoxymesterone, flutamide, foscamet, fumagillin analogs, fusidic acid, ganciclovir, gemcitabine HCL, gemtuzumab ozogamicin, gentamicin, glipizide, glutathione, glyburide, goserelin, gramicidin, heat shock proteins, heparin, herbimycon A, homatropine, humanized anti-IL-2receptor mAb (Daclizumab), hydrocortisone, hydroxyamphetamine, hydroxyurea, idoxuridine, ifosfamide, imidazole-based antifungals, insulin, interferon alfa-2a, interferon-gamma, interferons, interleukin-2, irinotecan HCL, ketoconazole, leflunomide, letrozole, leuprolide, levamisole, lidocaine, lipid formulations of antifungals, liposomalamphotericin B, lomustine, macrolide immunosuppressants, matrix metalloproteinase inhibitors, medroxyprogesterone, medrysone, melphalan, memantine, mercaptopurine, mestranol, metals (e.g., cobalt and copper), methapyriline, methazolamide, methotrexate, methylprednisolone, minocycline, mitomycin, mitotane, mitoxantrone hydrochloride, mono and polyclonal antibodies, muramyl dipeptide, mycophenolate mofetil, naphazoline, neomycin, nepafenac, neuroimmunophilin ligands, neurotrophic receptors(Aktkinase), neurotropins, nicotinamide (vitamin B3), nimodipine, nitrofurazone, nitrogen mustard, nitrosoureas, norethynodrel, NOS inhibitors, ondansetron, oprelvekin, oraptamers, oxytetracycline, paclitaxel, pentostatin, pheniramine, phenylephrine, phospholineiodine, pilocarpine, pipobroman, platelet factor 4, platinum coordination complexes (such as cisplatin and carboplatin), plicamycin, polymyxin, prednisolone, prednisone, procarbazine, tacrolimus, prophenpyridamine, prostaglandins, protamine, protease and integrase inhibitors, pyrilamine, rapamycin, ribavirin, rimexolone, rituximab, sargramostim, scopolamine, sodium propionate, streptozocin, succinic acid, sulfacetamide, sulfamethizole, sulfonamides, sulfoxazole, superoxide dismutase, suramine, tamoxifen, temozolomide, teniposide, tetracycline, tetrahydrazoline, thalidomide, thioguanine, thymopentin, thyroid hormones, tolazamide, tolbutamide, topotean hydrochloride, toremifene citrate, transforming factor beta2, trastuzumab, triamcinolone, triazole antifungals, trifluorothymidine, triptorelinpamoate, trisodium phosphonoformate, tropicamide, tumor necrosis factor, uracil mustard, valrubicin, VEGF antagonists (e.g., VEGF antibodies and VEGF antisense), vidarabine, vinblastine, vincristine, vindesine, vitamin B12 analogues, voriconazolerostaporfin, progranulin, taporfin sodium, MIRA-1 (Occulogix), Sirna-027 (Sirna Therapeutics Inc.), F200 (Protein Design Labs Inc), Cand5 (Acuity Pharmaceuticals), H8 (Cancervax Corporation), RetinoStat (Oxford Biomedica PLC), Angiotensin II Inhibitor (Genomed, Inc.), AK-1003 (Akom, Inc.), NX 1838 (Gilead Sciences Inc.), DL-8234 (Daiichi Pharmaceutical Co. Ltd), Envision TD (Control Delivery Systems, Inc.) and AMD Fab (Hoffmann-LaRoche).

In one embodiment, the drug is an anti-angiogenesis compound, e.g., for treatment of macular degeneration. Anti-angiogenesis compounds may exert their effects by any mechanism, including metalloproteinase inhibitors, monoclonal antibodies (e.g., anti-integrin or anti-VEGF antibodies), calcium channel inhibitors, vascular targeting agents, tetracycline derivatives, PKC inhibitors, IP-10 upregulators, growth factor antagonists, PDGF antagonists, VEGF antagonists, cytotoxics, antiproliferatives, and Na or Ca channel blockers. Exemplary anti-angiogenesis compounds include 2-methoxyestradiol (PANZEM) (EntreMed), A6, ABT-510, ABX-IL8 (Abgenix), actimid, Ad5FGF-4 (Collateral Therapeutics), AG3340 (Agouron Pharmaceuticals Inc. LaJolla, Calif.), alpha5beta1 integrin antibody, AMG001 (AnGes/Daichi Pharmaceuticals), anecortave acetate (Retaane, Alcon), angiocol, angiogenix (Endovasc Ltd), angiostatin (EntreMed), angiozyme, antiangiogenic antithrombin 3 (Genzyme Molecular Oncology), anti-VEGF (Genentech), anti-VEGF Mab, aplidine, aptosyn, ATN-161, avastin (bevacizumab), AVE8062A, Bay 12-9566 (Bayer Corp. West Haven, Conn.), benefin, BioBypass CAD (VEGF-121) (GenVec), MS275291, CAI (carboxy-anido imidazole), carboxymidotriazole, CC 4047 (Celgene), CC 5013 (Celgene), CC7085, CDC 801 (Celgene), Celebrex (Celecoxib), CEP-7055, CGP-41251/PKC412, cilengitide, CM101 (Carbomed Brentwood, Tenn.), col-3 (CollaGenex Pharmaceuticals Inc. Newton, Pa.), combretastatin, combretastatin A4P (Oxigene/Bristol-Myers Squibb), CP-547, 632, CP-564, 959, Del-1 (VLTS-589) (Valentis), dexrazoxane, didemin B, DMXAA, EMD 121974, endostatin (EntreMed), FGF (AGENT 3) (Berlex (Krannert Institute of Cardiology)), flavopiridol, GBC-100, genistein concentrated polysaccharide, green tea extract, HIF-1 alpha (Genzyme), human chorio-gonadotrophin, IM862 (Cytran), INGN 201, interferon alpha-2a, interleukin-12, iressa, ISV-120 (Batimastat), LY317615, LY-333531 (Eli Lilly and Company), Mab huJ591-DOTA-90 Yttrium (90Y), marimastat (British Biotech Inc. Annapolis, Md.), Medi-522, metaret (suramin), neoretna, neovastat (AEterna Laboratories), NM-3, NPe6, NV1FGF (Gencell/Aventis), octreotide, oltipraz, paclitaxel (e.g., taxol, docetaxel, or paxene), pegaptanib sodium (Eyetech), penicillamine, pentosan polysulphate, PI-88, prinomastat (Agouron Pharmaceuticals), PSK, psorvastat, PTK787/ZK222584, ranibizumab (Lucentis, Genentech), razoxane, replistatatin (Platelet factor-4), revimid, RhuMab, Ro317453, squalamine (Magainin Pharmaceuticals, Inc. Plymouth Meeting, Pa.), SU101 (Sugen Inc. Redwood City, Calif.), SU11248, SU5416 (Sugen), SU6668 (Sugen), tamoxifen, tecogalan sodium, temptostatin, tetrathiomol, tetrathiomolybdate, thalidomide (EntreMed Inc., Rockville, Md.), thalomid, TNP-470 (TAP Pharmaceuticals Inc. Deerfield, Wis.), UCN-01, VEGF (Genentech Inc. South San Francisco, Calif.), VEGF trap, Vioxx, vitaxin (Ixsys Inc. San Diego, Calif.), vitaxin-2 (MedImmune), ZD6126, and ZD6474. Additionally anti-angiogensis compounds found in vivo and suitable for use in the compositions and methods described herein include angiostatin (plasminogen fragment), metalloproteinase inhibitors (TIMPs), antiangiogenic antithrombin III (aaATIII), pigment epithelial-derived factor (PEDF), canstatin, placental ribonuclease inhibitor, cartilage-derived inhibitor (CDI), plasminogen activator inhibitor, CD59 complement fragment, platelet factor-4 (PF4), endostatin (collagen XVIII fragment), prolactin 16 kD fragment, fibronectin fragment, proliferin-related protein, gro-beta, retinoids, heparinases, tetrahydrocortisol-S, heparin hexasaccharide fragment, thrombospondin-1, human chorionic gonadotropin (hCG), transforming growth factor-beta, interferon alpha/beta/gamma, tumistatin, interferon inducible protein (IP-10), vasculostatin, interleukin-12 (IL-112), vasostatin (calreticulin fragment), kringle 5 (plasminogen fragment), angioarrestin, and 2-methoxyestradiol. Furthermore compounds that inhibit, block, or antagonize the angiogenic activity of the following species in vivo are useable in the methods and compositions described herein: angiogenin, placental growth factor, angiopoietin-1, platelet-derived endothelial cell growth factor (PD-ECGF), Del-1, platelet-derived growth factor-BB (PDGF-BB), fibroblast growth factors: acidic (aFGF) and basic (bFGF), pleiotrophin (PTN), follistatin, proliferin, granulocyte colony-stimulating factor (G-CSF), transforming growth factor-alpha (TGF-alpha), hepatocyte growth factor (HGF)/scatter factor (SF), transforming growth factor-beta (TGF-beta), interleukin-8 (IL-8), tumor necrosis factor-alpha (TNF-alpha), leptin, vascular endothelial growth factor (VEGF)/vascular permeability factor (VPF), midkine, progranulin, rostaporfin, taporfin sodium, MIRA-1 (Occulogix), Sirna-027 (Sirna Therapeutics Inc.), F200 (Protein Design Labs Inc), Cand5 (Acuity Pharmaceuticals), H8 (Cancervax Corporation), RetinoStat (Oxford Biomedica PLC), Angiotensin II Inhibitor (Genomed, Inc.), AK-1003 (Akom, Inc.), NX 1838 (Gilead Sciences Inc.), DL-8234 (Daiichi Pharmaceutical Co. Ltd), Envision TD (Control Delivery Systems, Inc.) and AMD Fab (Hoffmann-LaRoche).

Many drugs for the treatment of posterior segment disease may be inhibitors of ocular neovascularization. Inhibition may occur through the blocking or regulating of a number of pathways. These mechanisms may be intercellular or intracellular. For instance, the membrane-bound tyrosine kinase receptors VEGFR-1 and VEGFR-2 can be triggered by VEGF to result in activation of an intracellular tyrosine kinase domain and the resulting vascular endothelial cell proliferation. Drugs for the treatment of posterior segment disease may, for example, sequester and/or neutralize VEGF or block VEGFR-2. These drugs include, for example, VEGF-neutralizing oligonucleotide aptamers such as pegaptanib, humanized anti-VEGF monoclonal antibody fragments, such as ranibizumab, receptor analogs such as sFlt-1, and receptor-immunoglobulin fusion proteins. Other drugs may act as inhibitors of the tyrosine kinase signaling cascade or the degradation of VEGF messenger RNA with interfering RNA's. See van Wijngaarden et al, JAMA, Mar. 23/30, 2005, vol. 293, No. 12, pp 1509-1513.

In some embodiments, the drug being delivered can be or can include a nucleic acid. The nucleic acid may be, for example, RNA and/or DNA and may be single or double stranded. The nucleic acid component may include any number of base pairs, for example, from 1 to 100, 1 to 1000, 1 to 10,000, 1 to 100,000 or 1 to 1,000,000 base pairs. The nucleic acids may reduce or retard angiogenesis and may act by binding with or blocking receptor sites responsible for promoting angiogenesis. For example, the compound may be an RNA sequence that is an anti-sense antagonist of VEGF. The compound may react with a specific receptor site on the antagonist.

In other embodiments, the drug can be a compound that disrupts a metabolic pathway, for example, the metabolic pathways responsible for neovascular encroachment on the retina. This may include the disruption of enzymatic pathways in the posterior segment, such as occurs in diabetic retinopathy. The nucleic acid compounds may be intracellular or intercellular. In different embodiments, the anti-sense compound can interact with intracellular or intercellular molecules.

In one set of embodiments, an article can be used to introduce a drug for a posterior segment disease wherein the drug is a Vascular Endothelial Growth Factor (VEGF) ligand or ligand complex. The ligand or ligand complex may include any VEGF ligands and ligand complexes, such as, for example, those disclosed in U.S. Pat. No. 6,051,698, which is hereby incorporated by reference herein. These nucleic acid anti-angiogenesis compounds have been shown to be effective in treating, for example, macular degeneration. These compounds (the MACUGEN compounds) and their derivatives may be delivered directly from an article that is in contact with the eye and typically in contact with the cornea for extended periods of time (e.g., >1 hr).

As the compounds can be delivered passively from an article, e.g., a contact lens, and over an extended time, in some embodiments the compounds need not be derivitized and may consist of or consist essentially of nucleic acids. For example, the compounds may be void of fluoro groups such as 2′ fluoro groups, may be void of additional 2′ amino modification and may be void of 2′ O methyl modifications. The compounds may also include or be void of high molecular weight or lipophilic compounds that may, for instance, affect the in vivo stability of the compounds. Compounds may or may not include polyalkylene glycol and/or polyethylene glycol components. As the methods of administration described herein can provide, for example, a consistent concentration of drug directly to the eye over an extended period of time, some embodiments eliminate or reduce the need to alter the in vivo stability of the compounds. As nucleic acids are typically water soluble and soluble in isotonic saline, these compounds may be transferred into an article such as a hydrophilic contact lens by, for example, diffusion, or as a component of an aqueous solution that passes into the lens across an osmotic gradient.

In another embodiment, a protein or peptide, such as an anti-angiogenesis protein or peptide, may be delivered to the posterior segment via an article such as a contact lens. The protein may be an antibody or an antibody fragment. For example, another drug that may be used with the system is LUCENTIS (rhuFab V2), from Genentech, which is believed to be an anti-VEGF antibody fragment.

A drug may be admixed with a pharmaceutically acceptable carrier adapted to provide sustained release of the drug. Exemplary carriers include emulsions, suspensions, polymeric matrices, nanoparticles, microspheres, microcapsules, microparticles, liposomes, lipospheres, hydrogels, salts, and polymers with the drug reversibly bound electrostatically, chemically, or by entrapment. A pharmaceutically acceptable carrier may also include a transscleral diffusion promoting agent, such as dimethylsulfoxide, ethanol, dimethylformamide, propylene glycol, N-methylpyrolidone, oleic acid, isopropyl myristate, polar aprotic solvents, polar protic solvents, steroids, sugars, polymers, small molecules, charged small molecules, lipids, peptides, proteins, and surfactants. In other embodiments, a drug may be essentially free of a carrier such as a nanoparticle.

In some embodiments, the use of preservatives is non-ideal as they may transfer to a hydrogel at a disproportionately high concentration and cause cytotoxicity.

One example of a screening test that may be used to determine if a drug can be delivered by a contact lens, or similar, is to test the drug to determine its solubility in a hydrogel. A candidate drug should exhibit adequate aqueous solubility to be dispersed into a hydrophilic contact lens and to later diffuse or transfer from the contact lens to the ocular fluid. The drug should be soluble at a level that allows loading into the contact lens at a concentration adequate to produce an effect on the subject. For example, if a specific contact lens can hold 100 uL of solution and if a target loading level for the candidate drug is 5 nanograms per lens, then the solubility of the drug should be at least 5 ng/100 uL or 50 ng/mL.

If a candidate drug does not meet these solubility requirements, it may be derivitized to alter its solubility. Alternatively, surfactants and/or other solubility enhancers may be employed to improve the solubility of the drug.

Another technique that can be used to evaluate uptake and release of a drug is to expose an article, such as a lens, to a drug and then to evaluate the amount of uptake and release from the article using HPLC. For example, a lens loaded with a candidate drug can be placed in a solution such as artificial lachrymal fluid under ambient conditions. After a fixed period of time, eg, one hour, a sample of the fluid can be analyzed by HPLC to determine the amount of drug that has leached into the solution. Fresh solution can then be provided and additional samples may be analyzed at later times to develop a curve that indicates the amount of drug released over specific time intervals. From this data, one skilled in the art can determine peak dosing periods, overall dose rates and the expected lifetime of the loaded lens. This information can then be used, for example, to develop a loading target for a lens and a wearing schedule for the subject. Lens type can also be evaluated for use with specific drugs.

Similarly, the amount of uptake by a lens can be evaluated by placing a lens in a drug solution and monitoring, by periodic sampling, the amount of drug remaining in solution. Any reduction in drug concentration in the solution may be presumed to have been absorbed by the lens. This information can be used to determine, for example, concentrations and times that may be used for loading a lens with the drug.

In another embodiment, an article, for example a contact lens, can be used to deliver drugs effective for treating “dry eye” or “dry eye syndrome.” Traditionally, dry eye has been treated with the administration of artificial tears. While this treatment may ease symptoms and improve patient comfort, artificial tears do not treat the cause of the condition itself, that is, the inadequate production of lachrymal fluid by the subject. Recently, several drugs have been shown to be effective at treating dry eye. The procedures described herein provide an ideal method for delivering dry eye drugs as the drugs may be provided directly to the target and at a chosen concentration over a pre-determined period of time. For example, a dry eye drug may be delivered via a hydrophilic contact lens. The lens may be loaded with 1 microgram of a drug and a portion of that drug, for example, >50%, >75% or >90%, may be delivered to the eye over a 24 hour period. After delivery of the drug from the lens, the lens may be replaced with a fresh one or the lens may be reloaded with drug.

In some embodiments it may be notable that the drug is an active therapeutic that is delivered by the lens to a portion of the eye that is not in contact with the lens. In this way the drug acts at a site that is not in direct contact with the lens. This may serve, for example, to increase tear production in the subject rather than to simply replace missing lachrymal fluid. This is in contrast to a lens that is treated with a substance, such as a lubricant, e.g., petrolatum or PEG, that is designed to improve the feel of a contact lens on the eye.

The article, for example a lens, may also be used to deliver two or more drugs simultaneously. For example, a dry eye drug may be co-administered with a drug for a posterior segment condition. In another embodiment, two or more dry eye drugs can be co-administered. In another embodiment, a nucleic acid may be co-administered with a protein or polypeptide.

Administration of a dry eye drug via an article such as a contact lens may also ameliorate the dry eye condition by reducing moisture loss that occurs through evaporation. By forming a barrier between the surface of the eye and the air, the amount of surface area of the eye exposed to the air is reduced, resulting in a reduction in evaporative losses. Thus, the article may both deliver a dry eye drug as well as reduce evaporative moisture loss.

Any dry eye drug that can be loaded into or onto the lens may be delivered using this technique. Some of the therapeutic drugs with which the system may be useful include RESTASIS (cyclosporine ophthalmic emulsion), Diquafosol and salts thereof, such as Diquafosol tetrasodium, Rebamipide, OPC-12759, ELIDEL, pimecrolimus ophthalmic suspension, 15-HETE, hydroxyeicosatetraenoic acid, ECABET Sodium, prostaglandins, nicotinic acetylcholine receptor agonists, and phosphodiesterase inhibitors. Some of these compounds are described in U.S. Pat. Nos. 4,753,945, 6,277,855, 6,566,398, 6,645,978, 6,645,994, 6,659,985, and which are incorporated by reference herein.

Other drugs that help to relieve dry eye and may be useful with the invention include, for example, polyvinyl alcohol, hydroxypropyl methylcellulose, polyethylene glycol 400 castor oil emulsion, carboxymethylcellulose sodium, propylene glycol, hydroxypropyl guar, carboxymethylcelluose sodium, white petrolatum, mineral oil, dextran 70, glycerin, and hypromellose. Some other materials that may aid in the treatment of dry eye are flaxseed and fish oils, omega 3 and omega 6 fatty acids, lutein and primrose oil.

Treatment Approaches

To treat a posterior segment disease or dry eye condition, the hydrogels of the invention are contacted with the ocular fluid of an individual. The hydrogels may be employed in an open or closed eye period. When the article is shaped as a contact lens, the lens may simply be placed in the eye normally in order to deliver the drug. The hydrogel may also be part of a bandage or may be adhered (e.g., by adhesives or sutures) to the eye. If the hydrogel is placed internally in a patient, the hydrogel is advantageously biodegradable. The time period over which the lenses are worn may depend on the level of treatment desired or the amount of drug in the lens. Hydrogels may be considered to be disposable and may be replaced after a specified period of time, e.g., at least 0.5, 1, 1.5, 2, 2.5, 3, 3.5, 4, 4.5, 5, 7.5, 10, 15, or 24 hours. Alternatively, a hydrogel that has a depleted amount of drug may be recycled by soaking the hydrogel again in a solution of drug.

The methods of treatment described herein are capable of delivering a drug to the ocular environment of a patient for a period of time longer than the dwell time achievable by gels or drops. Furthermore, the concentration of the drug at the target may be more consistent over time as the rate of release from the hydrogel can be more controlled than from drops or gels. The convenience and simplicity of this system would in many cases enhance patient compliance with therapy. In addition, doubts about the amount of drug administered can be reduced or eliminated.

In certain embodiments, at least 0.001, 0.005, 0.01, 0.05, 0.1, 0.5, 1, 5, 10, 15, 20, 50, 75, 100, 200, 500, 750, or 1000 μg of the drug can be released from the hydrogel.

This delivery occurs by passive transfer and allows medications to be released into the ocular fluid. Drug concentrations at the target site, e.g., the retina or vitreous, may be maintained at +/−50%, +/−25% or +/−10% of a chosen level. The use of the invention may also allow patients to be treated using fewer applications than with traditional methods. In addition, the drug may be released from the hydrogel at a more rapid rate than the release of the drug into a fixed volume of fluid because as the eye produces tears, the drug released is flushed away from the site of application causing an increase in the relative rate of diffusion of the drug out of the hydrogel. The replenishing action of fluids such as tears may also effectively increase the rate of diffusion of the drug into the fluid and lead to earlier onset of therapeutic activity.

In one embodiment, the drug will penetrate the ocular tissue and migrate into the aqueous humor of the eye. Over time, the concentration of the drug will increase such that ocular tissue in the posterior segment of the eye will come into contact with the drug. The drug may have effects on other types of structures, cells, or tissues that may be present at the time of or prior to administration of the drug.

### EXAMPLE

To illustrate the ability to deliver a drug to the posterior segment using a hydrogel, an experiment was designed and completed using a contact lens to provide a drug to the retina. New Zealand White rabbits were treated with VEGF in each eye, followed by treatment with prednisolone in one eye, leaving the other as a control. VEGF is known to lead to edema in the retina and prednisolone is known to interfere with this mechanism. The contact lens was a high water ionic polymer lens (SOFTLENS 66, Bausch and Lomb, Rochester, N.Y.) having a water content of about 66%. Each lens had a diameter of about 13 mm.

Lens Preparation

Lenses were dessicated according to standard manufacturing procedures. Lenses were soaked at room temperature in a 1 mg/mL aqueous solution of VEGF (Sigma) for a period of 12 hours. Similar lenses were then separately soaked at room temperature in a 1 mg/mL aqueous solution of prednisolone for a period of 12 hours in order to load the lenses with the drug.

Lenses containing VEGF were placed on the cornea of each eye for a 4 hour closed-eye period. After removal of these lenses, a prednisolone loaded lens was then placed in the left eye for a 4 hour closed-eye period. The right eye was not treated with prednisolone.

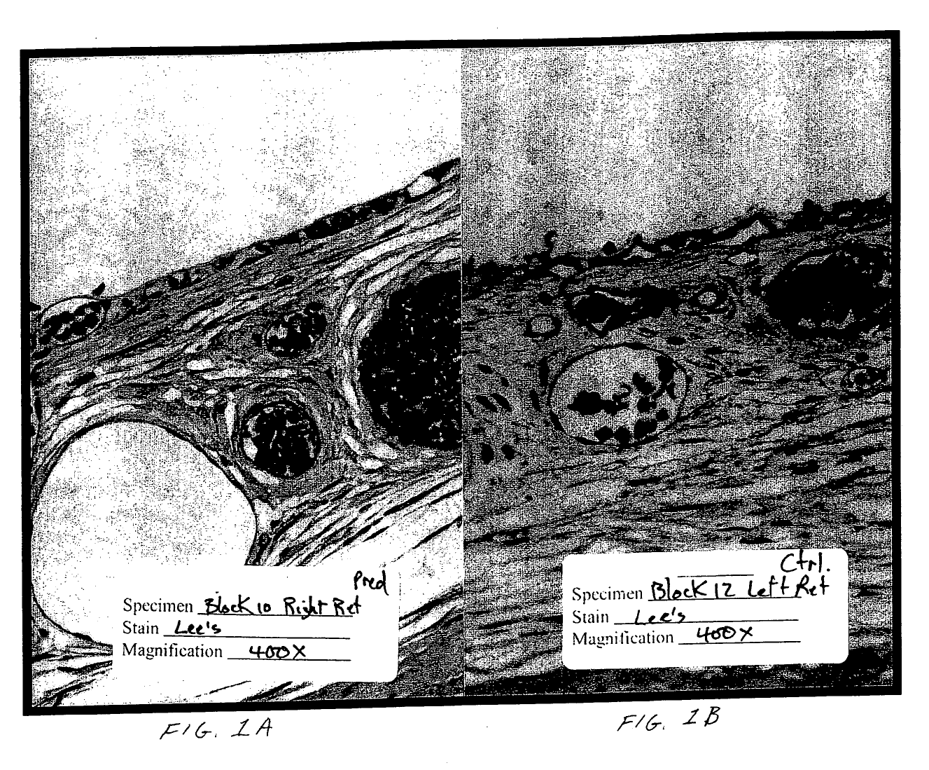

Within 48 hours, the respective retinas from each eye were harvested and cross-sectional slides were prepared using Lee's stain. Photomicrographs (400×) of the respective retinas are provided in FIGS. 1A and 1B. FIG. 1A shows the right retina that received VEGF but no prednisolone. FIG. 1B shows the left retina which received both VEGF and prednisolone. As is evident from the slides, the right retina (FIG. 1A no prednisolone) shows edema as evidenced by the large space that is not apparent in the left retina (FIG. 1B prednisolone). As both eyes were exposed to equal doses of VEGF, the lack of edema in the left retina must be the result of prednisolone being delivered from the lens to the retina. This occurs via the ocular circulatory system.

These results show the ability to deliver drugs to the posterior segment, and in particular to the retina, via a contact lens.

### Other Embodiments

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention.

Other embodiments are within the claims.

1. An article for treatment of posterior segment eye disease, the article comprising a substrate and an anti-angiogenesis nucleic acid drug wherein the article is capable of placement on the eye and the drug is capable of being passively released from the article.

2-29. (canceled)

30. The article of claim 1 wherein the nucleic acid is RNA.

31. The article of claim 1 wherein the RNA is at least one of transfer RNA, ribosomal RNA and/or messenger RNA.

32. The article of claim 1 wherein the nucleic acid is DNA.

33. The article of claim 1 wherein the nucleic acid comprises a Vascular Endothelial Growth Factor nucleic acid ligand.

34. The article of claim 1 wherein the drug consists essentially of a nucleic acid.

35. The article of claim 1 comprising a second drug.

36. The article of claim 1 wherein the second drug is a dry eye drug.

37. The article of claim 1 wherein the drug comprises an antagonist of macular degeneration or diabetic retinopathy.

38. The article of claim 37 wherein the drug comprises an anti-sense antagonist of Vascular Endothelial Growth Factor.

39. The article of claim 1 wherein the drug is transportable through the cornea or sclera or via the sinal cavity.

40-46. (canceled)

47. A method of treating a subject having or at risk of having posterior segment eye disease, the method comprising:
providing an article that comprises a substrate and an anti-angiogenesis nucleic acid drug;
placing the article in contact with the subject's eye; and
allowing the drug to be transported from the article to the eye.

48. The method of claim 47 comprising placing the article in contact with the subject's eye for at least one minute.

49-56. (canceled)

57. The method of claim 47 wherein the nucleic acid ligand is a non-naturally occurring nucleic acid.

58. The method of claim 47 wherein the compound consists essentially of a VEGF nucleic acid ligand.

59. The method of claim 47 wherein the drug is absorbed through the lymphatic system or the circulatory system of the eye.

60. The method of claim 47, wherein said posterior segment disease is selected from the group consisting of retinal detachment, neovascularization, diabetic retinopathy, macular degeneration, proliferative vitreoretinopathy, endophthalmitis, retinopathy of prematurity, posterior segment trauma, intraocular lens-related posterior segment complications, retinal vascular diseases, macular edema, intraocular tumors, retinal degeneration, vascular retinopathy, inflammatory diseases of the retina, AIDS-related retinitis, uveitis, and systemic diseases with retinal manifestations.

61. A method of treating a subject having or at a risk of having posterior segment eye disease, the method comprising administering to the subject a pharmaceutically effective quantity of a VEGF ligand consisting essentially of a nucleic acid.

62. The method of claim 61 wherein the nucleic acid comprises DNA

63. The method of claim 61 wherein the nucleic acid comprises RNA.

64. An article for therapeutic treatment of a dry eye condition, the article comprising a substrate and a drug wherein the article is capable of placement on the eye and the drug is capable of being passively released from the article.

65. The article of claim 64 wherein the drug comprises RESTASIS (cyclosporine ophthalmic emulsion), Diquafosol and salts thereof, Rebamipide, (OPC-12759, 2-(4-chlorobenzoylamino)-3-[2(1H)-quinolinon-4-yl]-propionic acid) ELIDEL (pimecrolimus ophthalmic suspension), 15-HETE (hydroxyeicosatetraenoic acid), ecabet sodium, prostaglandins, nicotinic acetylcholine receptor agonists, phosphodiesterase inhibitors, androgen and androgen analogs, Lipoxin A4 or inhibitors of acyl-CoA synthetase.

66. The method of claim 64 wherein the dry eye condition is keratoconjunctivitis sicca.

67. The article of claim 67 wherein a surface of the substrate is shaped to conform to a human cornea.

68. The article of claim 64 wherein the substrate comprises a hydrogel.

69. The article of claim 64, wherein said article is shaped as a contact lens.

70. The article of claim 69 wherein the contact lens is not a corrective lens.

71. The article of claim 69, wherein said article is capable of correcting vision.

72. The article of claim 68, wherein said hydrogel has a water content of between 10% and 90% by weight.

73. The article of claim 68, wherein said hydrogel has a water content of between 37.5% and 75% by weight.

74. The article of claim 64, wherein said article comprises a polymeric hydrogel.

75. The article of claim 74 wherein the hydrogel comprises a tetrapolymer of hydroxymethylmethacrylate, ethylene glycol, dimethylmethacrylate, and methacrylic acid.

76. The article of claim 64, wherein said drug is capable of being passively released into an ocular environment under ambient conditions.

77. The polymeric hydrogel of claim 74, wherein said hydrogel is capable of correcting vision in the range of +8.0 to −8.0 diopters.

78. The polymeric hydrogel of claim 74, wherein said hydrogel has a base curve between 8.0 and 9.0.

79. The article of claim 74, wherein said hydrogel comprises an ionic polymer.

80. The article of claim 74, wherein said hydrogel comprises a non-ionic polymer.

81. The polymeric hydrogel of claim 74, wherein said hydrogel comprises etafilcon A, vifilcon A, polymacon B, lidofilcon A, or vasurfilcon A.

82. The polymeric hydrogel of claim 74, wherein said hydrogel is at least partially absorbable in vivo.

83. The article of claim 82 wherein said hydrogel comprises a copolymer of trimethylene carbonate and polyglycolicacid, polyglactin 910, glyconate, poly-p-dioxanone, polyglycolic acid, polyglycolic acid felt, poly-4-hydroxybutyrate, a combination of poly(L-lactide) and poly(L-lactide-co-glycolide), glycol methacrylate, poly-DL-lactide, or Primacryl.

84. The article of claim 82, wherein said hydrogel comprises a composite of oxidized regenerated cellulose, polypropylene, and polydioxanone or a composite of polypropylene and poligelcaprone.

85. A method of treating a subject having or at risk of having a dry eye condition, the method comprising
providing an article that comprises a substrate and a dry eye drug;
placing the article in contact with the subject's eye; and
releasing the drug from the article.

86. The method of claim 85 wherein the article is in contact with the subject's eye for at least one minute.

87. The method of claim 85 wherein the dry eye condition is keratoconjunctivitis sicca.

88. The method of claim 85 wherein the drug comprises RESTASIS (cyclosporine ophthalmic emulsion), Diquafosol and salts thereof, Rebamipide, (OPC-12759, 2-(4-chlorobenzoylamino)-3-[2(1H)-quinolinon-4-yl]-propionic acid), ELIDEL (pimecrolimus ophthalmic suspension), 15-HETE (hydroxyeicosatetraenoic acid), ecabet sodium, prostaglandins, nicotinic acetylcholine receptor agonists, phosphodiesterase inhibitors, androgen and androgen analogs, Lipoxin A4 and inhibitors of acyl-CoA synthetase.

89. The method of claim 85 wherein the drug comprises polyvinyl alcohol, hydroxypropyl methylcellulose, polyethylene glycol 400 castor oil emulsion, carboxymethylcellulose sodium, propylene glycol, hydroxypropyl guar, carboxymethylcelluose sodium, white petrolatum, mineral oil, dextran 70, glycerin, hypromellose, flaxseed oil, fish oils, omega 3 and omega 6 fatty acids, lutein and primrose oil.
